(19) United States
(12) Patent Application Publication
(10) Pub. No.: US 2022/0040489 A1
(43) Pub. Date: Feb. 10, 2022

(54) SYSTEM AND METHOD FOR TREATING SCAR TISSUE OF A BIOLOGICAL SUBJECT USING RADIO FREQUENCY WAVES AND MANIPULATION

(71) Applicants: Bridget Witkowicz, Fall River (US); David Witkowicz, Fall River (US)

(72) Inventors: Bridget Witkowicz, Fall River (US); David Witkowicz, Fall River (US)

(21) Appl. No.: 17/391,955

(22) Filed: Aug. 2, 2021

Related U.S. Application Data
(60) Provisional application No. 63/061,016, filed on Aug. 4, 2020.

(51) Int. Cl. A61N 1/40; A61H 11/00; A61H 7/00

(52) U.S. Cl. CPC A61N 1/403; A61H 2201/10; A61H 7/003; A61H 11/00

(57) ABSTRACT

A system and method for treating tissue of a biological subject, including a radio frequency wave generator configured for generating a radio frequency wave at a therapeutically beneficial wavelength, a power supply configured for supplying power to the generator for generation of the wave, a controller configured for setting the wavelength, and a director configured for directing the wave to the tissue to achieve a therapeutically beneficial outcome for the subject. Preferably, the wave generates heat at a therapeutically beneficial temperature. Preferably, the director is configured for directing the wave to the tissue by placement of the director adjacent the tissue and movement of the director across the tissue in alternating general directions applied at various therapeutically beneficial speeds, various therapeutically beneficial distances, and various therapeutically beneficial time intervals within a desired treatment area of the tissue.

### FIELD OF THE INVENTION

The present general inventive concept relates generally to systems and methods for treating tissue of biological subjects, and more particularly to systems and method for treating scar tissue of biological subjects using radio frequency waves and manipulation.

### BACKGROUND OF THE INVENTION

The health of connective tissue has been found to directly affect pain levels, nerve sensitivity and freedom of movement. It has been estimated that over 90% of people with pain end up having connective tissue restrictions due to surgeries, old injuries, and trauma. In the past, such restrictions have not been recognized as important. However, it is now known that scar tissue develops adhesions and causes obstructions and consequently a decrease in flexibility, which in turn reduces blood flow, oxygen flow and toxic cell release. Muscles become choked and cannot perform at their best and as a result become sore and weak.

The body compensates, which often results in joint and tissue misalignment. Hips become shifted, backs are no longer straight, and shoulders are uneven. Pain sets in through the structure and cannot be validated by standard tests. When trauma, surgery and inflammation occur, restrictions of approximately 2,000 pounds per square inch on pain sensitive sites result and also cannot be validated by standard tests. This pressure acts like a bonded ligature on muscles and nerves and causes restriction of motion, and ultimately disease sets in.

Due to cell memory and muscle memory functions, the body sets into a lifelong pattern of distortion, which becomes labeled as chronic. When lack of movement sets in with pain over time, this causes “weak stimuli”, which increases physiologic activity and inhibits or abolishes normal activities.

There have previously been many systems and methods for treating tissue of biological subjects, including for treating scar tissue. These systems and methods have attempted to increase mobility, reduce pain, and reduce inflammation caused by scar tissue that has developed after surgery. While some have provided temporary relief, none have been able to address a significant underlying root cause of such side effects, which is the tangling of nerves at the affected tissue site. Therefore, these conventional solutions have not been effective for many patients and they have needed to rely on opioids for pain relief.

Accordingly, there is a need for a system and method that can reduce side effects of scar tissue by untangling nerves at a scar tissue site in a biological subject.

### SUMMARY OF THE INVENTION

The present general inventive concept provides a system and method for treating scar tissue of a biological subject using radio frequency waves and manipulation, which addresses the above-mentioned shortcomings by meeting the above described need.

### Objectives

An objective of the system and method of the present invention is to treat tissue of a biological subject using radio frequency waves and manipulation.

Another objective of the system and method of the present invention is to reduce side effects caused by scar tissue that has formed after surgery.

Yet another objective of the system and method of the present invention is to untangle nerves in scar tissue that has formed after surgery.

### General Overview and Benefits

The present general inventive concept provides a system and method for applying radio frequency waves and manipulation to scar tissue of a biological subject to provide relief from side effects of the scar tissue.

The system and method of the present invention provides for relief from symptoms of such side effects, including but not limited to lack of movement, pain, loss of sensation/feeling, edema, inflammation, and limited mobility.

The system and method of the present invention further provides for treating areas of the biological subject that have been wounded by surgery, overuse, tangled nerves, lack of movement from previous accidents, injuries and repetitive lifelong movements from work, sleep, sitting and strains. The system and method of the present invention further provides for improving the external appearance of scar tissue while treating the tissue internally.

The system and method of the present invention further provides for realigning jammed hips, addressing neck discomfort, tightening uneven shoulders, addressing body stiffness, addressing headaches, and addressing arthritis due to inflammation.

The system and method of the present invention provides relief by achieving various benefits, including but not limited to tissue release, adhesion release for proper oxygen flow, proper blood flow, nutrient release, toxic cell release, alignment, healing, soft tissue restructuring, tissue strengthening, restoring proper circulation to the wounded area, and causing the lymphatic system to release toxins that have been causing illnesses by being trapped.

The system and method of the present invention can be used to treat and address various aspects of biological subjects, including but not limited to soft tissue, scar tissue, connective tissue, skin, cutis, tegument, areolar tissue, nervous tissue, loose connective tissue, muscle tissue, connective tissue disease, connective tissue disorder, ground substance, gristle, cartilage, sarcoplasm, myofibril, myofibrilla, perineurium, endoneurium, tissue, connective, mastocyte, myofascial, granulation, mast cell, labrocyte, fascia, facia, matrix, granulation tumor, granulation, shaping, formation, adhesions, adhesion release, collagen, collagen renewal, tissue renewal, nerve release, tissue release, scar tissue release, elastic tissue, ligament, ligament release, submucosa, intercellular substance, connective tissue cells, connective tissue nevus, connective tissue proper, sheath, connective tissue sheath, tendon, and histocyte.

The objects, features, and advantages of the present invention, as well as the advantages thereof over existing prior art, which will become apparent from the descriptions herein, are accomplished by the improvements described in this specification and hereinafter described in the detailed description which fully discloses the invention but should not be considered as placing limitations thereon.

### Summary of an Example Embodiment

An example embodiment of the present invention can be summarized as providing a system and method for treating tissue of a biological subject, such as, for example, scar tissue that has formed after surgery, in order to provide relief from side effects caused by the scar tissue.

In the example embodiment, the system includes a radio frequency wave generator that generates a radio frequency wave at a therapeutically beneficial wavelength, a power supply that supplies power to the generator for generation of the wave, a controller that sets the wavelength, a director that directs the wave to the tissue, and a manipulator that manipulates the tissue. The manipulator has a rake shape so that it can be used to apply compressing and tension forces to the tissue in a raking motion to mechanically massage the tissue while the wave is applied. The director and the manipulator can be integrated into a single device that emits the wave during the manipulation of the tissue.

Further in the example embodiment, the wave is of a wavelength that generates heat at a therapeutically beneficial temperature.

In an example operation of the system and method, the director/manipulator is placed adjacent the tissue of the subject at the affected site and heat is applied to the tissue via the wave. During (or after) the application of the heat, the director/manipulator is moved across the tissue in a raking movement using rapid and semi-rapid short to long strokes. The treatment preferably is applied for approximately 10-15 minutes. Treatments preferably are repeated 1 to 3 times back-to-back once per week or twice per month, or more frequently depending on the type of tissue and the nature of the problem to be addressed. Treatments preferably can be reduced to less frequently (e.g., once per month or once every two months) as symptoms are alleviated.

Included below are descriptions of various characteristics of the manner in which the waves and manipulation can be applied.

Above has been briefly described a specific system and method of an example embodiment of the invention, for illustrative purposes only. The system and method of the invention will more generally be summarized below.

### Summary of the System of the Invention

In an aspect, the present general inventive concept provides a system for treating tissue of a biological subject.

In preferred embodiments, the system includes a radio frequency wave generator, a power supply, a controller, and a director. Preferably, the generator is configured for generating a radio frequency wave at a therapeutically beneficial wavelength, the power supply is configured for supplying power to the generator for generation of the wave, the controller is configured for setting the wavelength, and the director is configured for directing the wave to the tissue. Preferably, directing the wave to the tissue at the wavelength achieves a therapeutically beneficial outcome for the subject.

Further in preferred embodiments, the wave generates heat at a therapeutically beneficial temperature. Preferably, the temperature is one or more of inclusively between 35 and 55 degrees Celsius, 40 and 50 degrees Celsius, and 41 and 48 degrees Celsius. Preferably, the temperature is determined based on a depth of the tissue. Preferably, the temperature is determined based on a body part to which the tissue belongs.

Further in preferred embodiments, the director is configured for directing the wave to the tissue by placement of the director adjacent the tissue. Preferably, the wave penetrates the tissue to one or more of a depth of inclusively between 2 mm and 8 mm, and a depth of inclusively between 3 mm and 6 mm. Preferably, placement of the director adjacent the tissue includes maintenance of the director adjacent the tissue for a therapeutically beneficial time period. Further preferably, the time period is one or more of inclusively between 1 and 15 minutes, inclusively between 10 and 15 minutes, and inclusively between 3 and 5 minutes.

Preferably, the director is configured for directing the wave to the tissue by movement of the director across the tissue. Further preferably, the movement of the director across the tissue includes a plurality of movements across the tissue, one or more of the movements being applied in a direction, one or more others of the movements being applied in an other direction. Even further preferably, the plurality of movements across the tissue are applied at one of more of a therapeutically beneficial speed, a therapeutically beneficial distance, and a therapeutically beneficial time interval. Still further preferably, the therapeutically beneficial speed includes different speeds, the therapeutically beneficial distance includes different distances, and the therapeutically beneficial time interval includes different time intervals. Yet further preferably, the speeds, distances, and time intervals are determined based on at least one of a body part to which the tissue belongs and a treatment characteristic, wherein the characteristic is at least one of type and effectiveness.

Further preferably, the movement of the director across the tissue includes movements of alternating general direction applied at various therapeutically beneficial speeds, various therapeutically beneficial distances, and various therapeutically beneficial time intervals within a desired treatment area of the tissue.

Further in preferred embodiments, the director is further configured for manipulation of the tissue.

In certain preferred embodiments, the system further comprises a manipulator configured for manipulation of the tissue. Preferably, the manipulation is one or more of massage and separation. Preferably, the manipulator includes one or more of a flat shape, a rake shape, a claw shape and a comb shape.

Preferably, the manipulator is configured for manipulation of the tissue by application of force to the tissue. Further preferably, the force includes one or both of a compressive force and a tension force. Even further preferably, the force includes a compressive force and a tension force, the compressive force applied substantially toward a body of the subject and the tension force applied substantially perpendicular to the compression force. Still further preferably, the forces are applied in a plurality of raking movements across the tissue, one or more of the raking movements being applied in a direction, one or more others of the raking movements being applied in an other direction.

### Summary of the Method of the Invention

In another aspect, the present general inventive concept provides a method of treating tissue of a biological subject.

In preferred embodiments, the method includes providing a radio frequency wave generator, a power supply, a controller, and a director. Preferably, providing the generator includes generating a radio frequency wave at a therapeutically beneficial wavelength, providing the power supply includes supplying power to the generator for generation of the wave, providing the controller includes setting the wavelength, and providing the director includes directing the wave to the tissue.

Preferably, directing the wave to the tissue at the wavelength achieves a therapeutically beneficial outcome for the subject.

Further in preferred embodiments, generating the wave includes generating heat at a therapeutically beneficial temperature. Preferably, the temperature is one or more of inclusively between 35 and 55 degrees Celsius, 40 and 50 degrees Celsius, and 41 and 48 degrees Celsius. Preferably, the temperature is determined based on a depth of the tissue. Preferably, the temperature is determined based on a body part to which the tissue belongs.

Further in preferred embodiments, providing the director includes directing the wave to the tissue by placing the director adjacent the tissue. Preferably, the wave penetrates the tissue to a depth of inclusively between 3 mm and 6 mm. Preferably, placing the director adjacent the tissue includes maintaining the director adjacent the tissue for a therapeutically beneficial time period. Further preferably, the time period is one or more of inclusively between 1 and 15 minutes, inclusively between 10 and 15 minutes, and inclusively between 3 and 5 minutes.

Preferably, providing the director includes directing the wave to the tissue by moving the director across the tissue. Further preferably, moving the director across the tissue includes moving the director across the tissue in a plurality of movements, one or more of the movements being applied in a direction, one or more others of the movements being applied in an other direction. Even further preferably, the plurality of movements across the tissue are applied at one of more of a therapeutically beneficial speed, a therapeutically beneficial distance, and a therapeutically beneficial time interval. Still further preferably, the therapeutically beneficial speed includes different speeds, the therapeutically beneficial distance includes different distances, and the therapeutically beneficial time interval includes different time intervals. Yet further preferably, the speeds, distances, and time intervals are determined based on at least one of a body part to which the tissue belongs and a treatment characteristic, wherein the characteristic is at least one of type and effectiveness.

Further preferably, moving the director across the tissue includes moving the director across the tissue in alternating general directions and applying the director at various therapeutically beneficial speeds, various therapeutically beneficial distances, and various therapeutically beneficial time intervals within a desired treatment area of the tissue.

Further in preferred embodiments, providing the director includes manipulating the tissue.

In certain preferred embodiments, the method further comprises providing a manipulator, and providing the manipulator includes manipulating the tissue. Preferably, manipulating the tissue includes one or more of massaging the tissue and separating the tissue. Preferably, the manipulator includes one or more of a flat shape, a rake shape, claw shape and a comb shape. Preferably, providing the manipulator includes applying force to the tissue. Further preferably, the force includes one or both of a compressive force and a tension force. Even further preferably, applying the force includes applying a compressive force substantially toward a body of the subject and applying a tension force substantially perpendicular to the compression force. Still further preferably, applying the forces includes applying the forces in a plurality of raking movements across the tissue, one or more of the raking movements being applied in a direction, one or more others of the raking movements being applied in another direction.

It is briefly noted that upon a reading this disclosure, those skilled in the art will recognize various means for carrying out these intended features of the invention. As such it is to be understood that other methods, applications and systems adapted to the task may be configured to carry out these features and are therefore considered to be within the scope and intent of the present invention and are anticipated. With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

### BRIEF DESCRIPTION OF THE DRAWINGS

FIG. 1 illustrates a diagram of an example embodiment of a system of the present general inventive concept.

FIGS. 2-4 illustrate perspective, side and front views of a first type of a director of the system of the invention according to the example embodiment of the present general inventive concept.

FIGS. 5-7 illustrate perspective, side and front views of a second type of a director of the system of the invention according to the example embodiment of the present general inventive concept.

FIGS. 8-10 illustrate perspective, top and side views of a grounding plate of the system of the invention according to the example embodiment of the present general inventive concept.

FIGS. 11-13 illustrate perspective, side and bottom views of a manipulator of the system of the invention according to the example embodiment of the present general inventive concept.

FIG. 14 illustrates a flowchart of an example embodiment of a method of the present general inventive concept.

### DETAILED DESCRIPTION OF PREFERRED EMBODIMENTS

Reference will now be made in detail to exemplary embodiments of the present general inventive concept, examples of which are illustrated in the accompanying drawings, wherein like reference numerals refer to the like elements throughout. The exemplary embodiments are described below in order to explain the present general inventive concept by referring to the figures.

### Detailed Description of Preferred System Embodiments

In an aspect, the present general inventive concept provides a system for treating tissue of a biological subject. For example, the system can be used to treat scar tissue of a skin of a person.

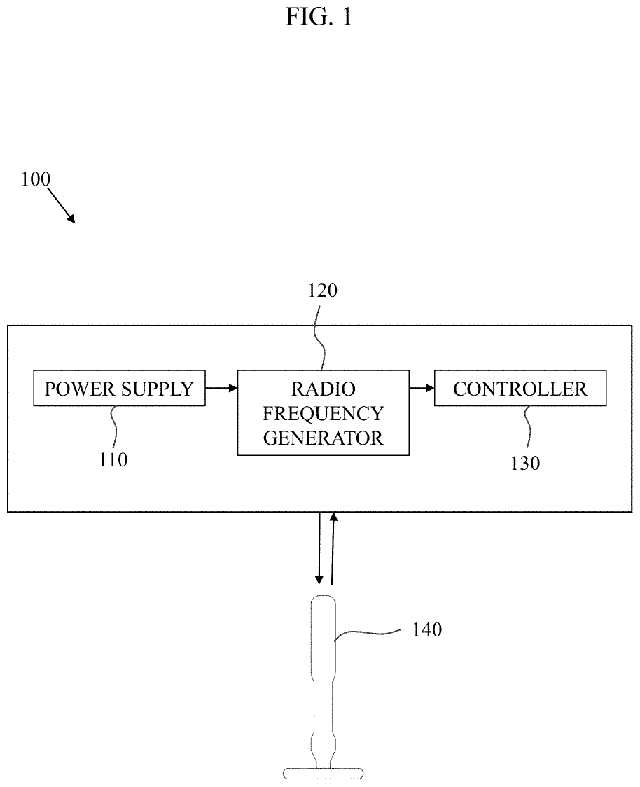

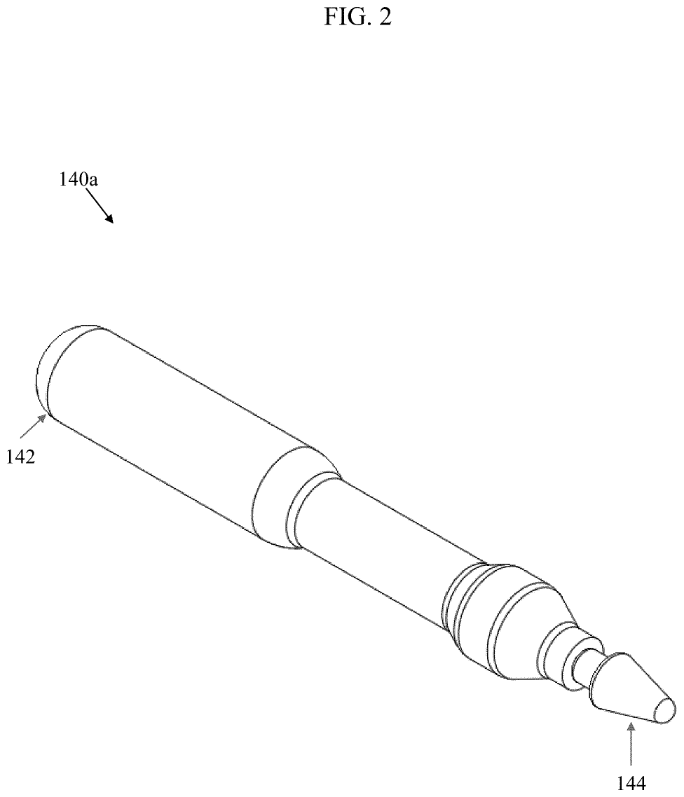

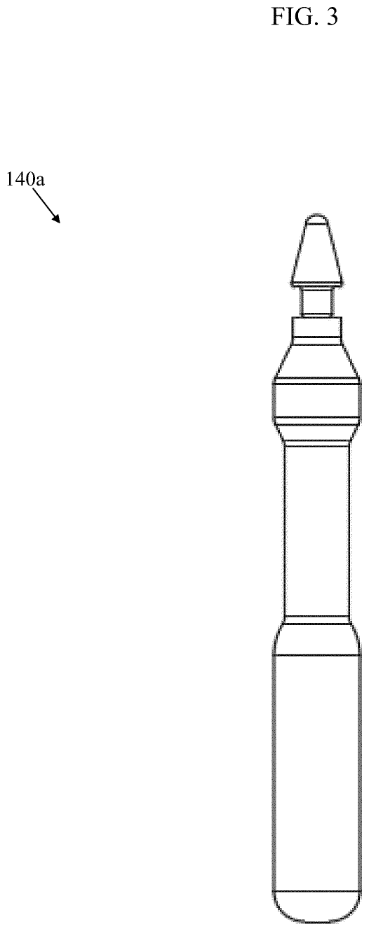

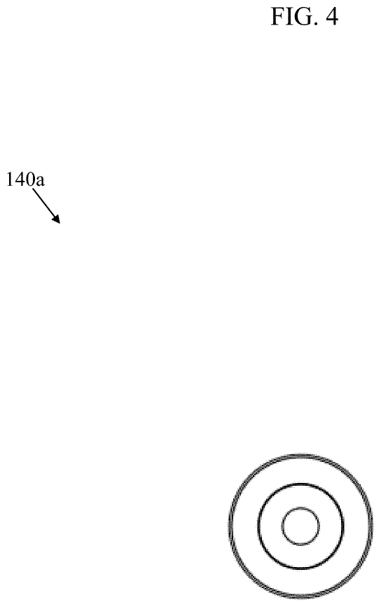

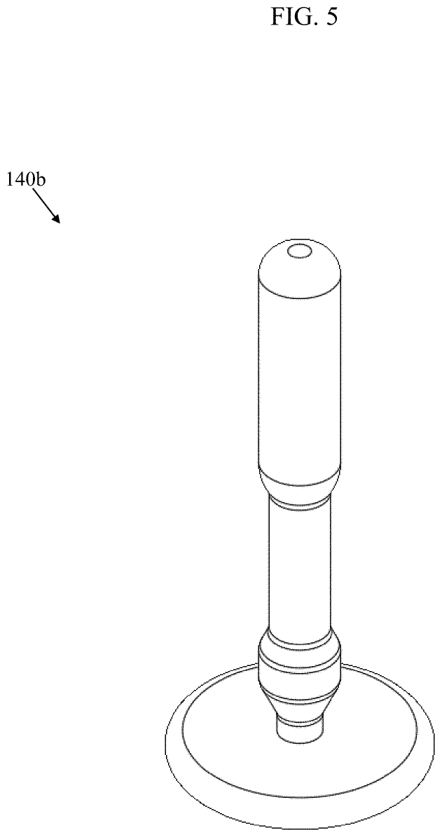

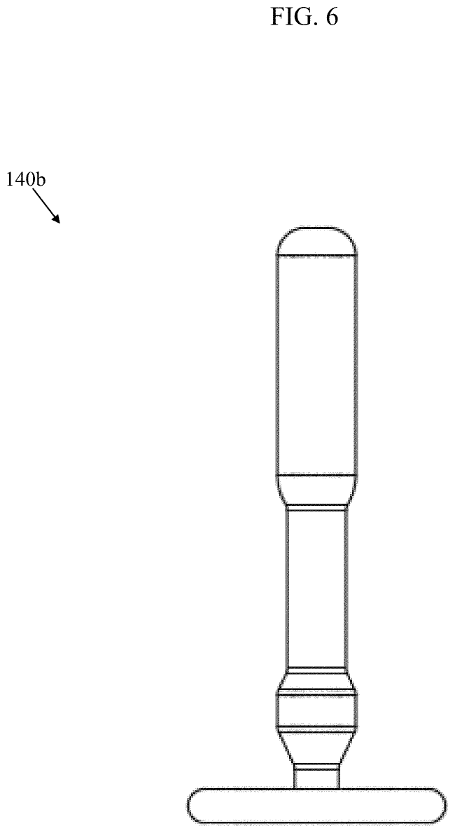

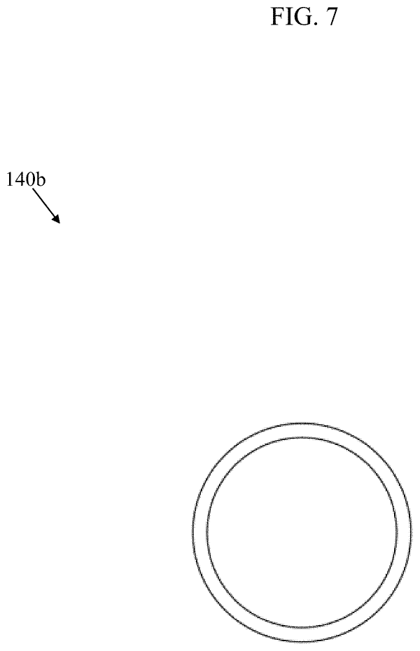

Referring now to FIG. 1, in preferred embodiments, the system 100 includes a radio frequency wave generator 120, a power supply 110, a controller 130, and a director 140. Preferably, the generator 120 is configured for generating a radio frequency wave at a therapeutically beneficial wavelength, the power supply 110 is configured for supplying power to the generator 120 for generation of the wave, the controller 130 is configured for setting the wavelength, and the director 140 is configured for directing the wave to the tissue. Referring additionally to FIGS. 2-7, the director 140 can be of any suitable shape, including but not limited to a first shape 140a with a narrow treatment end and a second shape 140b with a wide treatment end.

The director 140 includes a first end 142 designed for a user to hold the device and an opposing second end 144 designed to apply the treatment (wave) to the tissue. The director 140 may be formed in various shapes and sizes. For example, the director 140 may be formed in sizes including 2.0 inch width by 2.0 inch length, 4.0 inch width by 4.0 inch length, 6.0 inch width by 6.0 inch length, 7.0 inch width by 7.0 inch length, 8.0 inch width by 8.0 inch length, 9.0 inch width by 9.0 inch length, and 10.0 inch width by 10.0 inch length. The director 140 may include mono-polar, tri-polar, bi-polar options.

Preferably, directing the wave to the tissue at the wavelength achieves a therapeutically beneficial outcome for the subject. For example, the system can be used to generate and release radio frequency waves into the user's skin using the director 140.

Further in preferred embodiments, the wave generates heat at a therapeutically beneficial temperature. For example, the radio frequency waves can be of a wavelength that generates heat.

Preferably, the temperature is one or more of inclusively between 35 and 55 degrees Celsius, 40 and 50 degrees Celsius, and 41 and 48 degrees Celsius. However, any temperature determined to be therapeutically beneficial can be used. In certain contemplated uses, the temperature can be set initially and then modified to other temperatures during the treatment time, and/or across multiple or different treatment sessions. Preferably, the temperature is determined based on a depth of the tissue. For example, in certain contemplated uses, if the affected area of the tissue is deeper than expected or initially determined, then (1) the initial temperature setting can be increased (e.g., by approximately one degree Celsius to approximately five degrees Celsius) and/or (2) the application of the heat can be repeated for additional time (e.g., for an additional approximately 3 minutes to an additional 5 minutes). Preferably, the temperature is determined based on a body part to which the tissue belongs.

Further in preferred embodiments, the director 140 is configured for directing the wave to the tissue by placement of the director 140 adjacent the tissue. For example, the director can be placed on the skin directly, or on clothing adjacent the skin, or held at a distance from the skin or from clothing adjacent the skin. For example, the distance from the skin and the presence of any material between the director and the skin can vary according to the desired treatment and can vary over the course of the treatment. Preferably, the wave penetrates the tissue to one or more of a depth of inclusively between 2 mm and 8 mm, and a depth of inclusively between 3 mm and 6 mm. Preferably, placement of the director 140 adjacent the tissue includes maintenance of the director 140 adjacent the tissue for a therapeutically beneficial time period. Further preferably, the time period is one or more of inclusively between 1 and 15 minutes, inclusively between 10 and 15 minutes, and inclusively between 3 and 5 minutes.

Preferably, the director 140 is configured for directing the wave to the tissue by movement of the director across the tissue. Further preferably, the movement of the director 140 across the tissue includes a plurality of movements across the tissue, one or more of the movements being applied in a direction, one or more others of the movements being applied in another direction. Even further preferably, the plurality of movements across the tissue are applied at one of more of a therapeutically beneficial speed, a therapeutically beneficial distance, and a therapeutically beneficial time interval. Still further preferably, the therapeutically beneficial speed includes different speeds, the therapeutically beneficial distance includes different distances, and the therapeutically beneficial time interval includes different time intervals. Yet further preferably, the speeds, distances, and time intervals are determined based on at least one of a body part to which the tissue belongs and a treatment characteristic, wherein the characteristic is at least one of type and effectiveness.

Further preferably, the movement of the director 140 across the tissue includes movements of alternating general direction applied at various therapeutically beneficial speeds, various therapeutically beneficial distances, and various therapeutically beneficial time intervals within a desired treatment area of the tissue.

For example, the director can be placed on the subject's skin and moved using long and short strokes, back and forth over an affected area. It should be understood that the director can be moved at any speed, including but not limited to rapid, semi-rapid, and slow strokes. It should further be understood that the director can be moved at any length of stroke, including but not limited to short and long strokes.

For example, if a subject has developed scar tissue on his or her back, the director can be moved from the subject's lower back, from hip to hip, right to left in approximately 5 to 7 second and across back and forth for approximately 15 minutes. It should be understood that the specific movements described herein, and their timing are merely examples, and movements of any sequence, pattern, type and timing are contemplated by the invention, and further can be determined based on any criteria, including but not limited to any characteristic of the subject, the subject's condition, the anticipated effectiveness and determination of a trained professional.

Preferably, if necessary, a lubricant such as, for example, mineral oil, can be applied to the director to provide smooth movements between the director and the subject.

Further in preferred embodiments, the director 140 is further configured for manipulation of the tissue.

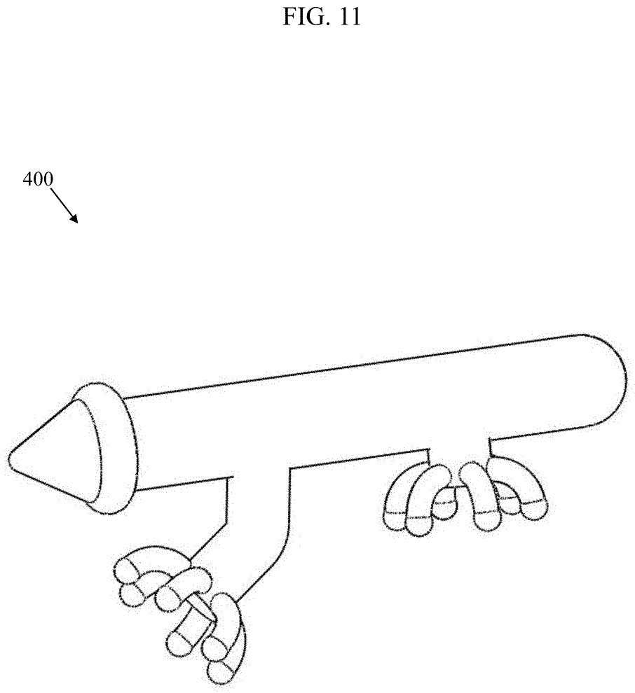

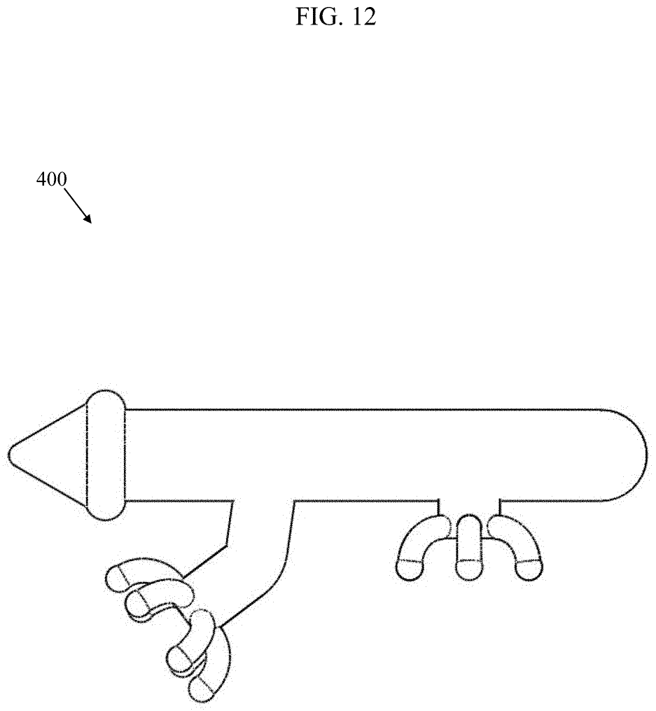

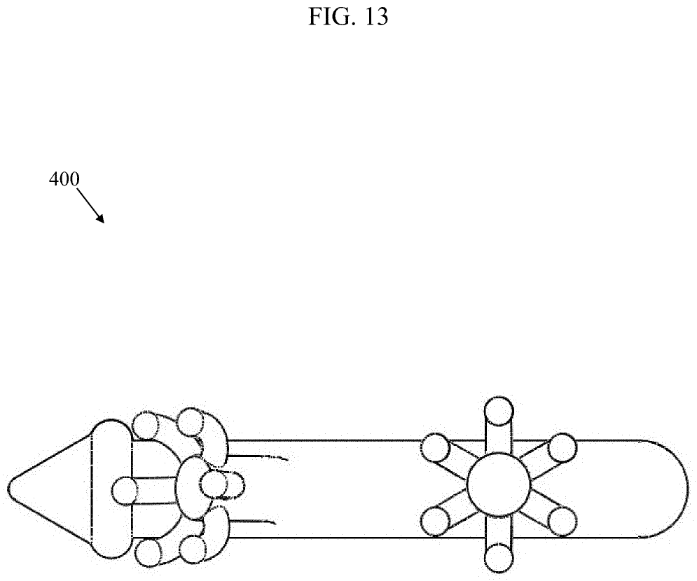

Referring additionally to FIGS. 11-13, in certain preferred embodiments, the system further comprises a manipulator 400 configured for manipulation of the tissue.

Preferably, the manipulation is one or more of massage and separation. Preferably, the manipulator 400 includes one or more of a flat shape, a rake shape, a claw shape (as shown) and a comb shape. For flat shapes, a round shape having a diameter of 2 inches, a round shape having a diameter of 4 inches, an oblong shape having a large diameter of 3 inches and a short diameter of 2 inches, and an oblong shape having a large diameter of 5 inches and a short diameter of 3.25 inches, are each preferred for respective applications. Preferably, on flat shapes or other shapes, one or more tips (e.g., preferably rounded) can extend from the surface (e.g., preferably by ¼ inch or ½ inch) for enhanced mechanical manipulation of tissue. Preferably, the shapes are included on interchangeable components for ease of switching between them for different applications. Preferably, the manipulator 400 is formed from one or more materials including but not limited to ceramic, metal, plastic, wood, glass and polymer, and combinations thereof.

Preferably, the manipulator 400 is configured for manipulation of the tissue by application of force to the tissue. Further preferably, the force includes one or both of a compressive force and a tension force. Even further preferably, the force includes a compressive force and a tension force, the compressive force applied substantially toward a body of the subject and the tension force applied substantially perpendicular to the compression force. Still further preferably, the forces are applied in a plurality of raking movements across the tissue, one or more of the raking movements being applied in a direction, one or more others of the raking movements being applied in another direction.

For example, the director (at least when configured for manipulation) or the manipulator can include (1) a fascial rake shape made of plastic and configured for mechanical massage; (2) a flat shape made of ceramic and configured for separation of tissue; or (3) a claw shape or comb shape and configured for releasing adhesions from tissue under the subject's skin. For example, releasing adhesions from tissue under the subject's skin can be accomplished using the manipulator by performing a combing motion. For example, combing and/or raking motions performed back and forth in an affected area (e.g., from hip to hip of a subject) can cause tissue knots to become untangled in the area.

The present general inventive concept is designed and/or configured to first apply heat/radio frequency wave to tissue and then to claw/comb the tissue to improve muscle atrophy, muscle asphyxiation, nerve tissues (i.e., pinched, damaged, and residuals from illnesses such as Bells Palsy, stroke, scoliosis). The present general inventive concept is also designed and/or configured to improve chocked tissue adhesions from damage from accidents, surgeries, diseases, and various illnesses. However, the present general inventive concept is not limited thereto.

Preferably, if a subject experiences pain during treatment, the force used to apply the manipulation can be reduced as necessary.

Preferably, if desired, a hot towel can be placed over the treated area to reinforce the treatment.

Preferably, if desired, treatments described herein can be repeated between one and three times to increase their effectiveness.

It should be understood that the director (at least when configured for manipulation) or the manipulator can include or be formed in any shape and of any material, and that the shapes and materials described herein are merely examples.

It should further be understood that the forces applied can be of any safe magnitude, any direction, at any speed, and at any interval, and that the forces and applications thereof are merely examples.

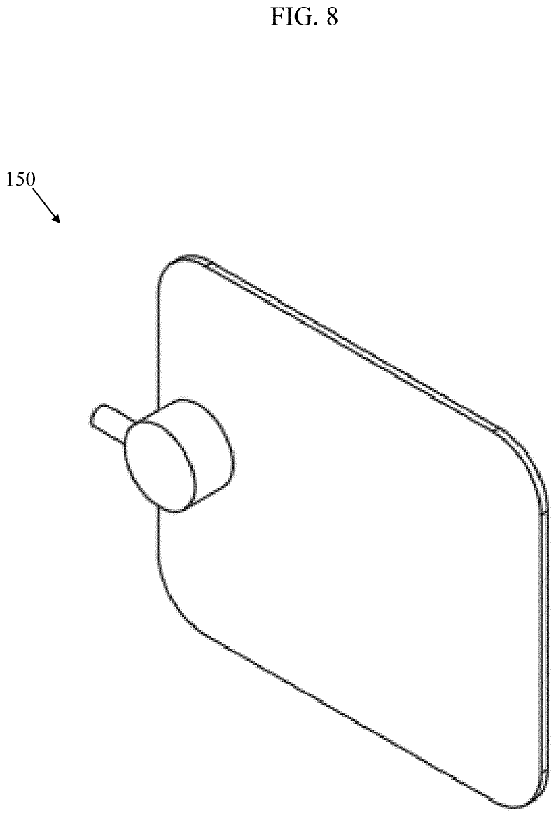

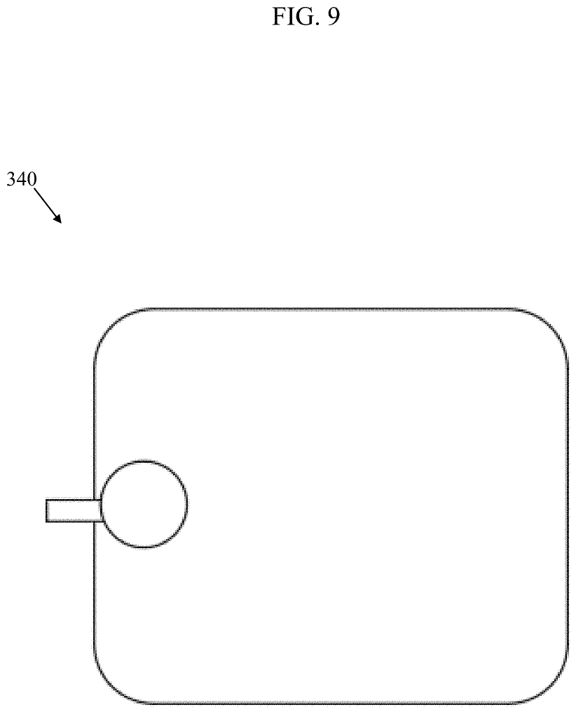

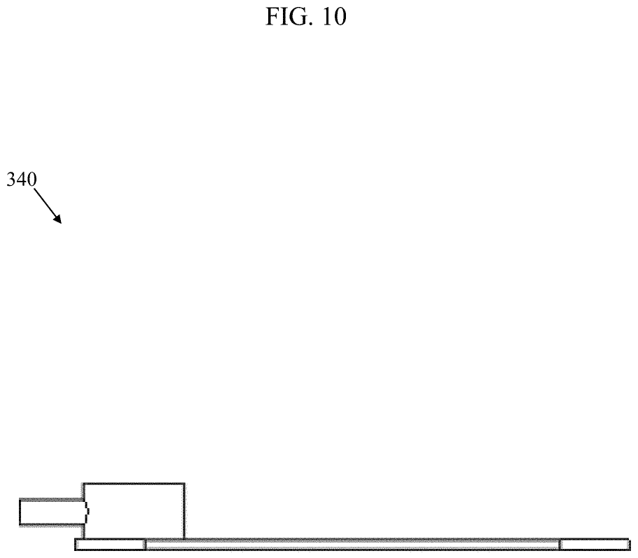

Referring additionally to FIGS. 8-10, in these and other preferred embodiments, the system further includes a grounding pad 150 configured for grounding the subject prior to directing the wave to the tissue. However, it should be understood that depending on the treatment needed and the frequencies used, grounding the subject may be achieved by other means without use of a grounding pad, or may not be necessary.

### Detailed Description of Preferred Method Embodiments

In another aspect, the present general inventive concept provides a method of treating tissue of a biological subject. For example, as discussed above, the system can be used to treat scar tissue of a skin of a person.

The following discussion of a method of the invention can be understood in reference to the system components discussed above and their described use, however, it should be understood that the method of the invention can be practiced not only with such system components, but also with additional or other components, without departing from the scope of the invention, and that such components are merely example of components that can be used to carry out the method of the invention.

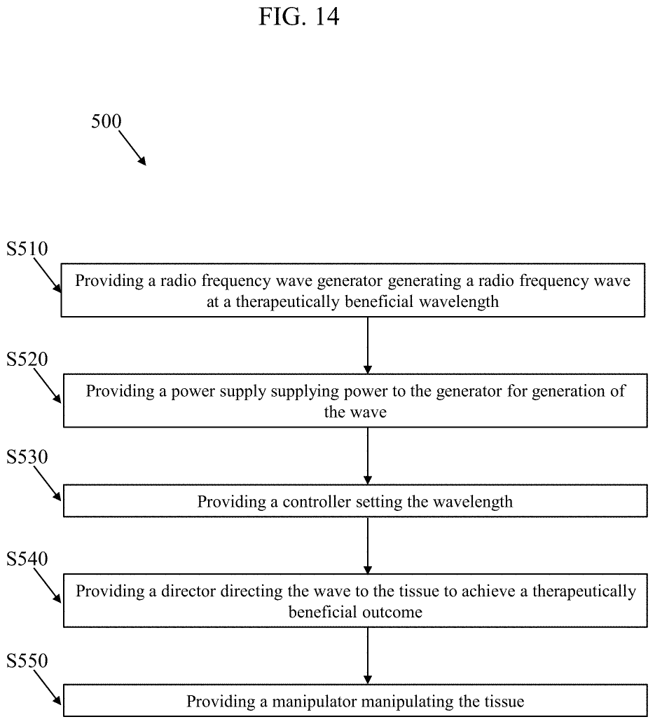

Referring now to FIG. 14, in preferred embodiments, the method 500 includes providing a radio frequency wave generator (S510), a power supply (S520), a controller (S530), and a director (S540). Preferably, providing the generator includes generating a radio frequency wave at a therapeutically beneficial wavelength, providing the power supply includes supplying power to the generator for generation of the wave, providing the controller includes setting the wavelength, and providing the director includes directing the wave to the tissue. As discussed above, the director can be of any suitable shape, including but not limited to a shape with a narrow treatment end and a shape with a wide treatment end.

Preferably, directing the wave to the tissue at the wavelength achieves a therapeutically beneficial outcome for the subject. For example, as discussed above, the system can be used to generate and release radio frequency waves into the user's skin using the director.

Further in preferred embodiments, generating the wave includes generating heat at a therapeutically beneficial temperature. For example, as discussed above, the radio frequency waves can be of a wavelength that generates heat.

Preferably, the temperature is one or more of inclusively between 35 and 55 degrees Celsius, 40 and 50 degrees Celsius, and 41 and 48 degrees Celsius. However, as discussed above, any temperature determined to be therapeutically beneficial can be used. In certain contemplated uses, the temperature can be set initially and then modified to other temperatures during the treatment time, and/or across multiple or different treatment sessions. Preferably, the temperature is determined based on a depth of the tissue. For example, as discussed above, in certain contemplated uses, if the affected area of the tissue is deeper than expected or initially determined, then (1) the initial temperature setting can be increased (e.g., by approximately one degree Celsius to approximately five degrees Celsius) and/or (2) the application of the heat can be repeated for additional time (e.g., for an additional approximately 3 minutes to an additional 5 minutes). Preferably, the temperature is determined based on a body part to which the tissue belongs.

Further in preferred embodiments, providing the director includes directing the wave to the tissue by placing the director adjacent the tissue. For example, as discussed above, the director can be placed on the skin directly, or on clothing adjacent the skin, or held at a distance from the skin or from clothing adjacent the skin. For example, the distance from the skin and the presence of any material between the director and the skin can vary according to the desired treatment and can vary over the course of the treatment. Preferably, the wave penetrates the tissue to one or more of a depth of inclusively between 2 mm and 8 mm, and a depth of inclusively between 3 mm and 6 mm. Preferably, placing the director adjacent the tissue includes maintaining the director adjacent the tissue for a therapeutically beneficial time period. Further preferably, the time period is one or more of inclusively between 1 and 15 minutes, inclusively between 10 and 15 minutes, and inclusively between 3 and 5 minutes.

Preferably, providing the director includes directing the wave to the tissue by moving the director across the tissue. Further preferably, moving the director across the tissue includes moving the director across the tissue in a plurality of movements, one or more of the movements being applied in a direction, one or more others of the movements being applied in another direction. Even further preferably, the plurality of movements across the tissue are applied at one of more of a therapeutically beneficial speed, a therapeutically beneficial distance, and a therapeutically beneficial time interval. Still further preferably, the therapeutically beneficial speed includes different speeds, the therapeutically beneficial distance includes different distances, and the therapeutically beneficial time interval includes different time intervals. Yet further preferably, the speeds, distances, and time intervals are determined based on at least one of a body part to which the tissue belongs and a treatment characteristic, wherein the characteristic is at least one of type and effectiveness.

Further preferably, moving the director across the tissue includes moving the director across the tissue in alternating general directions and applying the director at various therapeutically beneficial speeds, various therapeutically beneficial distances, and various therapeutically beneficial time intervals within a desired treatment area of the tissue.

For example, as discussed above, the director can be placed on the subject's skin and moved using long and short strokes, back and forth over an affected area. It should be understood that the director can be moved at any speed, including but not limited to rapid, semi-rapid, and slow strokes. It should further be understood that the director can be moved at any length of stroke, including but not limited to short and long strokes.

For example, as discussed above, if a subject has developed scar tissue on his or her back, the director can be moved from the subject's lower back, from hip to hip, right to left in approximately 5 to 7 second and across back and forth for approximately 15 minutes. It should be understood that the specific movements described herein and their timing are merely examples, and movements of any sequence, pattern, type and timing are contemplated by the invention, and further can be determined based on any criteria, including but not limited to any characteristic of the subject, the subject's condition, the anticipated effectiveness and determination of a trained professional.

Preferably, as discussed above, if necessary, a lubricant such as, for example, mineral oil, can be applied to the director to provide smooth movements between the director and the subject.

Further in preferred embodiments, providing the director includes manipulating the tissue.

In certain preferred embodiments, the method 500 further comprises providing a manipulator (S550), and providing the manipulator includes manipulating the tissue. Preferably, manipulating the tissue includes one or more of massaging the tissue and separating the tissue. Preferably, the manipulator includes one or more of a flat shape, a rake shape, a claw shape and a comb shape. For flat shapes, a round shape having a diameter of 2 inches, a round shape having a diameter of 4 inches, an oblong shape having a large diameter of 3 inches and a short diameter of 2 inches, and an oblong shape having a large diameter of 5 inches and a short diameter of 3.25 inches, are each preferred for respective applications. Preferably, on flat shapes or other shapes, one or more tips (e.g., preferably rounded) can extend from the surface (e.g., preferably by ¼ inch or ½ inch) for enhanced mechanical manipulation of tissue. Preferably, the shapes are included on interchangeable components for ease of switching between them for different applications. Preferably, providing the manipulator includes applying force to the tissue. Further preferably, the force includes one or both of a compressive force and a tension force. Even further preferably, applying the force includes applying a compressive force substantially toward a body of the subject and applying a tension force substantially perpendicular to the compression force. Still further preferably, applying the forces includes applying the forces in a plurality of raking movements across the tissue, one or more of the raking movements being applied in a direction, one or more others of the raking movements being applied in another direction.

In alternative embodiments, the manipulator includes an LED light (e.g., red, blue, green, and yellow) that is applied/emitted to the tissue through the manipulator to improve muscle atrophy, muscle asphyxiation, nerve tissues (i.e., pinched, damaged, and residuals from illnesses such as Bells Palsy, stroke, scoliosis).

For example, as discussed above, the director (at least when configured for manipulation) or the manipulator can include (1) a fascial rake shape made of plastic and configured for mechanical massage; (2) of a flat shape made of ceramic and configured for separation of tissue; or (3) a claw shape or comb shape and configured for releasing adhesions from tissue under the subject's skin. For example, as discussed above, releasing adhesions from tissue under the subject's skin can be accomplished using the manipulator by performing a combing motion. For example, as discussed above, combing and/or raking motions performed back and forth in an affected area (e.g., from hip to hip of a subject) can cause tissue knots to become untangled in the area.

Preferably, as discussed above, if a subject experiences pain during treatment, the force used to apply the manipulation can be reduced as necessary.

Preferably, as discussed above, if desired, a hot towel can be placed over the treated area to reinforce the treatment.

Preferably, as discussed above, if desired, treatments described herein can be repeated between one and three times to increase their effectiveness.

It should be understood, as discussed above, that the director (at least when configured for manipulation) or the manipulator can include or be formed in any shape and of any material, and that the shapes and materials described herein are merely examples.

It should further be understood, as discussed above, that the forces applied can be of any safe magnitude, any direction, at any speed, and at any interval, and that the forces and applications thereof are merely examples.

In these and other preferred embodiments, the method 500 can further include grounding the subject prior to directing the wave to the tissue. However, it should be understood that depending on the treatment needed and the frequencies used, grounding the subject may not be necessary.

### Experimental Details

During development of the invention, clinically significant increases in, and restoration of, movement and decreases in, and even eliminations of, pain were achieved using the application of radio frequency waves and the manipulation of the affected tissue. In some cases, the results were realized nearly instantaneously. In other cases, the results were realized over time. The positive results effectively created new cell memories.

In a first case, a six-year-old subject was experiencing pain from a shoulder injury and was considering hip surgery as her hips were also experiencing pain from the shoulder injury. Prior to treatment in accordance with the present invention, pain was evaluated at a 6 on a 10-point scale and mobility was at 40% of normal range of motion. After two treatments in accordance with the present invention, pain was evaluated at a 1 on a 10-point scale and considered a “nagging ache”, and mobility was at 100% of normal range of motion. After a third treatment in accordance with the present invention, the pain was gone and full range of motion was maintained. Further, the subject no longer considered the hip surgery to be necessary.

In a second case, a retail manager who works on her feet more than 9 hours each day was experiencing pain evaluated at a 12 on a 10-point scale and numbness and frequent loss of feeling around her foot more than 1 year after foot surgery. Surgeons recommended cortisone shots as the only option for addressing the symptoms. After two treatments in accordance with the present invention applied 3 weeks apart, and a third treatment in accordance with the present invention applied 4 weeks thereafter, pain was completely gone and full feeling had returned to the foot.

In a third case, a powerlifter who underwent back surgery that fused intervertebral disc levels 14 and 15 subsequently felt pain, lost feeling in her foot and had difficulty with balance, and accordingly was diagnosed with dropped foot and required use of a cane. The surgeon offered cortisone shots, physical therapy, and a boot for the dropped foot, as the only course of treatment. After treatment in accordance with the present invention, at the surgical site and out to the hips, the pain was eliminated, even during activities such as bending over and powerlifting. Further, the subject no longer needed a cane, realized she never had dropped foot, and regained her balance and all feeling in her foot. After the first treatment, she could again feel her back and hips, regained 70% of her mobility, and walked out without a cane. After the second treatment, her pain was eliminated, she had regained 100% of her mobility, and felt only 30-40% numbness in her lower leg. After the third treatment, her pain remained eliminated, she had retained 100% of her mobility, felt only 20% numbness in her lower leg, and enjoyed full balance on both feet.

In a fourth case, a subject had severe sciatic pain for three years until the pain could be tolerated only if the subject was on all fours for multiple days on end. The treating physician prescribed steroids. Prior to treatment in accordance with the present invention, the subject experienced complete loss of feeling in his foot and lower leg, reduced balance, daily pain evaluated at 8 to 9 on a 10-point scale, mobility at 30-40% of normal, and numbness at 60%. After treatment in accordance with the present invention, including aligning the tissue in the hip area, feeling in the foot and lower leg was restored to 90-100%, balance was restored, pain was gone, mobility was restored to 90-100% of normal, and numbness decreased to 10%. The first two treatments in accordance with the present invention were applied three weeks apart, and then a third treatment in accordance with the present invention was applied one month later.

In a fifth case, a subject had suffered with scoliosis from age 7 to her current age 30, experiencing limiting movement and spasms. Two titanium rods were installed in her back to provide support. She had undergone surgery, massage therapy, physical therapy, and many magnetic resonance imaging studies, seeking relief and strength, all to no avail. She had decided to forego having children for fear of not being able to support the weight of carrying them to term or later being able to hold them on her hips. After only one treatment in accordance with the present invention, applied to her shoulders, middle of her back and up to her neck, she experienced relief from her condition for the first time in her life, enjoying increased movement and fewer spasms, allowing her to work and pursue college courses. In addition, she felt as though she could change her mind about having children.

In a sixth case, a subject suffered from numbness on the side of his face, and limited mobility. Computer aided tomography scans and blood work showed no apparent cause for these conditions. He received no answers after two emergency room visits and a consultation with his general practitioner. After one treatment in accordance with the present invention, he experience reduced numbness.

In other cases, clients with scars from cancer received treatments in accordance with the present invention for aesthetics reasons, with successful results including reducing the external appearance of the scar tissue, separating the internal adhesions, and loosening tightening, and relieving pain.

### Other Embodiments

It is additionally noted and anticipated that although the system and method is shown in a simple form, various components and aspects of the system and method may be differently shaped or slightly modified when forming the invention herein. As such those skilled in the art will appreciate the descriptions and depictions set forth in this disclosure or merely meant to portray examples of preferred modes within the overall scope and intent of the invention, and are not to be considered limiting in any manner. While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention.

What is claimed is:

1. A system for treating tissue of a biological subject, comprising
a radio frequency wave generator;
a power supply;
a controller; and
a director; wherein
the generator is configured for generating a radio frequency wave at a therapeutically beneficial wavelength;
the power supply is configured for supplying power to the generator for generation of the wave;
the controller is configured for setting the wavelength;
the director is configured for directing the wave to the tissue; and
directing the wave to the tissue at the wavelength achieves a therapeutically beneficial outcome for the subject.

2. The system of claim 1, wherein the wave generates heat at a therapeutically beneficial temperature.

3. The system of claim 2, wherein the temperature is one or more of inclusively between 35 and 55 degrees Celsius, 40 and 50 degrees Celsius, and 41 and 48 degrees Celsius.

4. The system of claim 2, wherein the temperature is determined based on a depth of the tissue.

5. The system of claim 1, wherein the director is configured for directing the wave to the tissue by placement of the director adjacent the tissue.

6. The system of claim 5, wherein the wave penetrates the tissue to one or more of a depth of inclusively between 2 mm and 8 mm, and a depth of inclusively between 3 mm and 6 mm.

7. The system of claim 5, wherein placement of the director adjacent the tissue includes maintenance of the director adjacent the tissue for a therapeutically beneficial time period.

8. The system of claim 7, wherein the time period is one or more of inclusively between 1 and 15 minutes, inclusively between 10 and 15 minutes, and inclusively between 3 and 5 minutes.

9. The system of claim 5, wherein the director is configured for directing the wave to the tissue by movement of the director across the tissue.

10. The system of claim 9, wherein the movement of the director across the tissue includes a plurality of movements across the tissue, one or more of the movements being applied in a direction, one or more others of the movements being applied in another direction.

11. The system of claim 10, wherein the plurality of movements across the tissue are applied at one of more of a therapeutically beneficial speed, a therapeutically beneficial distance, and a therapeutically beneficial time interval.

12. The system of claim 11, wherein the therapeutically beneficial speed includes different speeds, the therapeutically beneficial distance includes different distances, and the therapeutically beneficial time interval includes different time intervals.

13. The system of claim 9, wherein the movement of the director across the tissue includes movements of alternating general direction applied at various therapeutically beneficial speeds, various therapeutically beneficial distances, and various therapeutically beneficial time intervals within a desired treatment area of the tissue.

14. The system of claim 1, wherein the director is further configured for manipulation of the tissue.

15. The system of claim 1, further comprising a manipulator configured for manipulation of the tissue.

16. The system of claim 15, wherein the manipulator includes one or more of a flat shape, a rake shape, a claw shape and a comb shape.

17. The system of claim 15, wherein the manipulator is configured for manipulation of the tissue by application of force to the tissue.

18. The system of claim 17, wherein the force includes one or both of a compressive force and a tension force.

19. The system of claim 18, wherein the force includes a compressive force and a tension force, the compressive force applied substantially toward a body of the subject and the tension force applied substantially perpendicular to the compression force.

20. The system of claim 19, wherein the forces are applied in a plurality of raking movements across the tissue, one or more of the raking movements being applied in a direction, one or more others of the raking movements being applied in another direction.
